(19) United States
(12) Patent Application Publication
(10) Pub. No.: US 2021/0121848 A1
(43) Pub. Date: Apr. 29, 2021

(54) 3D CAGE TYPE HIGH NITROGEN CONTAINING MESOPOROUS CARBON NITRIDE FROM DIAMINOGUANIDINE PRECURSORS FOR CO2 CAPTURE AND CONVERSION

(71) Applicant: SABIC Global Technologies B.V., Bergen op Zoom (NL)

(72) Inventors: Kripal S. LAKHI, Mawson Lakes (AU); Dae-Hwan PARK, Mawson Lakes (AU); Ugo RAVON, Thuwal (SA); Khalid ALBAHILY, Thuwal (SA); Jessica SCARANTO, Thuwal (SA); Ajayan VINU, Mawson Lakes (AU)

(21) Appl. No.: 16/617,391

(22) PCT Filed: May 22, 2018
(86) PCT No.: PCT/IB2018/053621

§ 371 Date: Nov. 26, 2019

Related U.S. Application Data
(60) Provisional application No. 62/513,732, filed on Jun. 1, 2017.

(51) Int. Cl. B01J 20/02; C01B 21/06; B01J 20/28; B01J 20/30; C07C 29/15; B01D 53/62; B01D 53/86; B01D 53/81

(52) U.S. Cl. CPC B01J 20/02; B01D 2255/9207; B01J 20/28019; B01J 20/28083; B01J 20/28061; B01J 20/28071; B01J 20/28073; B01J 20/3057; B01J 20/3078; B01J 20/3071; C07C 29/15; B01D 53/62; B01D 53/8671; B01D 53/8693; B01D 53/81; C01P 2004/32; C01P 2006/17; C01P 2006/12; C01P 2006/14; C01P 2006/32; C01P 2002/72; C01P 2004/03; C01P 2002/82; B01D 2253/102; B01D 2253/308; B01D 2253/306; B01D 2253/311; B01D 2257/504; B01D 2255/702; B01D 2255/9202; C01B 21/0605

(57) ABSTRACT

Certain embodiments of the invention are directed to nitrogen rich three dimensional C₃N₄+ mesoporous graphitic carbon nitride (gMCN) material formed from diaminoguanidine precursors, the gMCN having a spherical morphology and an average monomodal pore diameter between 6.5 to 9.5 nm.

### CROSS REFERENCE TO RELATED APPLICATIONS

This application claims the benefit of priority of U.S. Provisional Patent Application No. 62/513,732 filed Jun. 1, 2017, which is hereby incorporated by reference in its entirety.

### BACKGROUND OF THE INVENTION

### A. Field of the Invention

The invention generally concerns a composition or catalyst for carbon dioxide capture. In particular the composition or catalyst includes a nitrogen rich three-dimensional mesoporous graphitic carbon nitride (3D gMCN) that provides for carbon dioxide adsorption and/or activation.

### B. Description of Related Art

It is well known that carbon dioxide (CO₂) emissions are at least partially responsible for global warming. One strategy for decreasing CO₂ emissions into the atmosphere is to use CO₂ containing emissions as feedstock for other processes, thus utilizing the CO₂ instead of releasing it into the atmosphere. For this reason, many researchers have tried to activate or capture the CO₂ molecule through the use of various materials. However, due to the high stability of this molecule, CO₂ activation is extremely challenging, which oftentimes results in inefficient catalytic activity.

A class of mesoporous carbon nitride (MCN) materials has been considered for potential application in the fields of catalysis, gas adsorption, and energy conversion due to their unique electronic, optical, and basic properties (Lakhi et al., Chem. Soc. Rev., 2016; Wang et al., Nat. Mater., 2009, 8:76; Zheng et al., Energy Environ. Sci., 2012, 5:6717). The synthesis of MCNs has been realized via a templating approach using mesoporous silica as a sacrificial template. Recently, researchers have reported the development of various structural and textural properties for high surface areas, different pore sizes, uniform morphology, as well as the control of surface functionalities, nitrogen content, and band gaps and positions (Talapaneni et al., ChemSusChem, 2012, 5:700; Jin et al., Angew. Chem. Int. Ed., 2009, 48:7884; Zhong et al., Sci. Rep., 2015, 5:12901; Lakhi et al., RSC Adv., 2015, 5:40183; Chinese Patent Publication No. 204326446 to Jie et al.; and Li et al. Nano Res., 2010, Vol. 3, pp. 632-642).

Although the reported MCN materials have shown textural features useful for various catalytic performances and gas adsorption capacities; however, the use of these materials for CO₂ activation remains elusive. The currently available MCN materials typically have relatively low catalytic activity, which severely hinders the commercial scalability of such materials.

### SUMMARY OF THE INVENTION

gMCN materials of the current invention provide a solution to the adsorption and catalysis problems associated with CO₂ capture and activation. In particular, improved nitrogen rich 3D cage type C₃N₄₊ gMCN (e.g., a gMCN having a N/C ratio of greater than 1.33) having with ordered mesostructure and high surface area prepared from a silica template using a high nitrogen containing diaminoguanidine as a single molecule carbon and nitrogen precursor have been developed for capture and/or activation of CO₂. By way of example, the inventors have discovered a process to produce the gMCN material, which results in the material having appropriate structural characteristics that enhance CO₂ sequestration and/or activation. Without wishing to be bound by theory, it is believed that the use of diaminoguanidine based precursors results in 3D C₃N₄₊ gMCN materials having suitable surface area, pore diameters, and/or activity to capture CO₂ from a liquid or gas stream.

Certain embodiments are directed to a nitrogen rich three-dimensional graphitic mesoporous carbon nitride (gMCN) material formed from diaminoguanidine (e.g., 1,3-diaminoguanidine) precursors the gMCN having (i) a spherical morphology, (ii) a C₃N₄₊ stoichiometry, and (iii) a monomodal distribution of pores having an average pore diameter between 6.5 to 9.5 nm. In certain aspects the material has a nitrogen to carbon (N/C) ratio of about 1.45 to 1.6. The material can have a BET surface area of 180 to 200 m²/g, preferably 190 to 198 m²/g. In other aspects the material has a total pore volume of 0.4-0.7 cm³/g, preferably 0.5 cm³/g. The CO₂ adsorption capacity of the material can be 7.0 to 9.5 mmol/g at 273K and 30 bar. In certain aspects the isosteric heat of adsorption of the material is 12 to 80 kJ/mol, 14.9-45.4 kJ/mol, or 30-80 kJ/mol. The material can be a negative replica of a FDU-12 silica template.

Other embodiments are directed to methods of synthesizing a three dimensional carbon nitride material from a diaminoguanidine (e.g., 1,3-diaminoguanidine) precursor comprising: (a) contacting a silica template with an aqueous diaminoguanidine precursor solution forming a templated reaction mixture; (b) heating the templated reaction mixture to a temperature between 40 and 200° C., preferably between 80 and 120° C. for 4 to 8 hours forming a first heated reaction mixture; (c) heating the first heated reaction mixture to a temperature between 100 and 200° C., preferably between 140 to 180° C., preferable 160° C., for 4 to 8 hours forming a second heated reaction mixture; (d) carbonizing the second heated reaction mixture by heating to about 300 to 500° C., preferably 400° C., for 4 to 6 hours forming a template/diaminoguanidine-based carbon nitride product; and (e) removing the template forming a nitrogen rich three-dimensional C₃N₄₊ graphitic mesoporous carbon nitride (gMCN) material formed from diaminoguanidine. In certain aspects the silica template is a FDU-12 silica template. In a further aspect the diaminioguanidine precursor is 1,3-diaminoguanidine. The template reaction mixture can be heated at a temperature of about 130° C. forming a FDU-12-130 template. In certain aspects template reaction mixture is heated at a temperature of about 150° C. forming a FDU-12-150 template. The methods can further include crushing the second heated reaction mixture prior to the carbonizing. The method can further include bringing the second heated mixture to carbonization temperature using a ramping rate of 2 to 4° C./min. In certain aspects carbonizing is performed under constant nitrogen flow. In certain aspects the first heated reaction mixture can be incubated at a temperature of 130° C. or 150° C., including any value or range there between. The template can be removed by treating the template/diaminoguanidine-based carbon nitride product with hydrogen fluoride or an ethanol wash.

In certain aspects the silica template is formed by methods including one or more of the following steps: (f) adding tetraethylorthosilicate (TEOS) to a mixture of F-127 surfactant, potassium chloride (KCl), 1,3,5-trimethylbenzene, and hydrogen chloride (HCl) forming a template reaction mixture; (g) incubating the template reaction mixture at a temperature of about 30 to 40° C., preferably 35° C. for 1 to 4 hours; (h) heating the template reaction mixture to 100-200° C. for 1 to 4 days forming a heated template reaction mixture; (i) drying the heated template reaction mixture at 100° C. for 5 to 10 hours forming a dried template reaction mixture; and (j) calcining the dried template reaction mixture at a temperature of 500 to 600° C., preferably 540° C., forming a FDU-12 template.

Other aspects of the invention are directed to a CO₂ capture process that includes contacting a nitrogen rich three-dimensional C₃N₄₊ graphitic mesoporous carbon nitride (gMCN) of the present invention formed from diaminoguanidine precursors, the gMCN having a monomodal pore distribution with an average pore diameter between 6.5 to 9.5 nm, with a CO₂ containing feed source, wherein CO₂ is absorbed in or to gMCN. The process can further include incubating the CO₂ absorbed gMCN under conversion conditions forming a CO₂ conversion product. In certain aspects the CO₂ conversion product includes methanol.

In the context of the present invention 21 embodiments are described. Embodiment 1 is 1 nitrogen rich three-dimensional graphitic mesoporous carbon nitride (gMCN) material having (i) a spherical morphology, (ii) a C₃N₄₊ stoichiometry where the nitrogen to carbon (N/C) ratio from 1.45 to 1.6, and (iii) an average pore diameter between 6.5 to 9.5 nm. Embodiment 2 is the material of embodiment 1, wherein N/C is 1.5. Embodiment 3 is the material of embodiment 1 or 2, wherein the gMCN is formed from templated diaminoguanidine, preferably 1,3-diaminoguanidine. Embodiment 4 is the material of any one of embodiments 1 to 3, wherein the material has a BET surface area of 180 to 200 m²/g, preferably 190 to 198 m²/g. Embodiment 5 is the material any one of embodiments 1 to 4, wherein the material has a total pore volume of 0.4-0.7 cm³/g, preferably 0.5 cm³/g. Embodiment 6 is the material any one of embodiments 1 to 5, wherein the material has a CO₂ adsorption capacity of 7.0 to 9.5 mmol/g at 273K and 30 bar. Embodiment 7 is the material of any one of embodiments 1 to 6, wherein the material has an isosteric heat of adsorption of 10, 15, 20, 25, 30, 35 to 40, 45, 50, 55, 60, 65, 70, 75, 80 kJ/mol. Embodiment 8 is the material any one of embodiments 1 to 7, wherein the material is a negative replica of a FDU-12 silica template.

Embodiment 9 is a method of synthesizing a three dimensional carbon nitride material formed from a diaminoguanidine precursor comprising: (a) contacting a silica template with an aqueous diaminoguanidine precursor solution forming a templated reaction mixture; (b) heating the templated reaction mixture to a temperature between 40 and 200° C., preferably between 80 and 120° C. for 4 to 8 hours forming a first heated reaction mixture; (c) heating the first heated reaction mixture to a temperature between 100 and 200° C., preferably between 140 to 180° C., preferable 160° C., for 4 to 8 hours forming a second heated reaction mixture; (d) carbonizing the second heated reaction mixture by heating to about 300 to 500° C., preferably 400° C., for 4 to 6 hours forming a template/1,3-diaminoguanidine-based carbon nitride product; and (e) removing the template forming nitrogen rich three-dimensional C₃N₄₊ graphitic mesoporous carbon nitride (gMCN) material formed from the diaminoguanidine. Embodiment 10 is the method of embodiment 9, wherein the silica template is formed by: (f) adding tetraethyl orthosilicate (TEOS) to a mixture of F-127 surfactant, potassium chloride (KCl), 1,3,5-trimethylbenzene, and hydrogen chloride (HCl) forming a template reaction mixture; (g) incubating the template reaction mixture at a temperature of about 30 to 40° C., preferably 35° C. for 1 to 4 hours; (h) heating the template reaction mixture to 100 to 200° C. for 1 to 4 days forming a heated template reaction mixture; (i) drying the heated template reaction mixture at 100° C. for 5 to 10 hours forming a dried template reaction mixture; and (j) calcining the dried template reaction mixture at a temperature of 500 to 600° C., preferably 540° C., forming a FDU-12 silica template. Embodiment 11 is the method of any one of embodiments 9 to 10, wherein the template reaction mixture is heated at a temperature of about 130° C. forming a FDU-12-130 template. Embodiment 12 is the method of any one of embodiments 9 to 10, wherein the template reaction mixture is heated at a temperature of about 150° C. forming a FDU-1-150 template. Embodiment 13 is the method of any one of embodiments 9 to 12, further comprising crushing the second heated reaction mixture prior to the carbonizing. Embodiment 14 is the method of any one of embodiments 9 to 13, further comprising bringing the second heated mixture to carbonization temperature using a ramping rate of 2 to 4° C./min. Embodiment 15 is the method of any one of embodiments 9 to 14, wherein carbonizing is performed under constant nitrogen flow. Embodiment 16 is the method of embodiment 9, wherein the first heated reaction mixture is held at a temperature of 130° C. Embodiment 17 is the method of embodiment 9, wherein the first heated reaction mixture is held at a temperature of 150° C. Embodiment 18 is the method of any one of embodiments 9 to 17, wherein the template is removed by treating the template/diaminoguanidine-based carbon nitride product with hydrogen fluoride or an ethanol wash.

Embodiment 19 is a CO₂ capture process comprising contacting a nitrogen rich three-dimensional C₃N₄₊ graphitic mesoporous carbon nitride (gMCN) of any one of claims 1 to 8, with a CO₂ containing feed source, wherein CO₂ is absorbed in or to gMCN. Embodiment 20 is the process of claim 19, further comprising incubating the CO₂ absorbed gMCN under conversion conditions forming a CO₂ conversion product. Embodiment 21 is the process of embodiment 20, wherein the CO₂ conversion product comprises methanol.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The term “substantially” and its variations are defined to include ranges within 10%, within 5%, within 1%, or within 0.5%.

The terms “inhibiting” or “reducing” or “preventing” or any variation of these terms includes any measurable decrease or complete inhibition to achieve a desired result.

The term “effective,” as that term is used in the specification and/or claims, means adequate to accomplish a desired, expected, or intended result.

The terms “wt. %,” “vol. %,” or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The gMCN materials and processes of making and using these materials of the present invention can “comprise,” “consist essentially of,” or “consist of” particular ingredients, components, compositions, method steps, etc., disclosed throughout the specification. With respect to the transitional phase “consisting essentially of,” in one non-limiting aspect, a basic and novel characteristic of the gMCN materials of the present invention are their ability to efficiently adsorb and/or activate CO₂.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Low and wide angle (inset) XRD patterns of FD-T-DAMG.

FIG. 2. N₂ adsorption desorption isotherms and pore size distribution (inset) of FD-T-DAMG samples.

FIGS. 3A-3B. (FIG. 3A) HR-SEM imaging shows spherical shaped morphology of FD-130-DAMG (P,Q) and FD150-DAMG (R,S) samples. (FIG. 3B) HR-TEM images showing the presence of mesochannels (A1, A2) FD-130-DAMG and (B1, B2) FD-150-DAMG

FIG. 4. FT-IR spectra of FD-130-DAMG sample.

FIGS. 5A-5C. (FIG. 5A) Survey spectra of FD-T-DAMG samples. (FIG. 5B) High resolution N1s spectra of FD-T-DAMG samples. (FIG. 5C) High resolution C1s spectra of FD-T-DAMG samples.

FIGS. 6A-6B. (FIG. 6A) C K-edge, and (FIG. 6B) N K-edge NEXAFS spectra of the (a) FD150-DAMG and (b) non-porous g-C₃N₄ prepared by dicyandiamide at 550° C.

FIG. 7. CO₂ adsorption isotherms of FD-T-DAMG samples at 273 K and 30 bar.

FIGS. 8A-8B. CO₂ adsorption isotherms at 273 K and 283 K and pressure up to 30 bar (FIG. 8A) FD130-DAMG and (FIG. 8B) FD150-DAMG.

FIGS. 9A-9B. Isosteric heat of adsorption calculated using isotherms at 273 K and 283 K (FIG. 9A) FD-130-DAMG, and (FIG. 9B) FD150-DAMG.

FIG. 10. Low angle XRD patterns of FDU-12-T silica template.

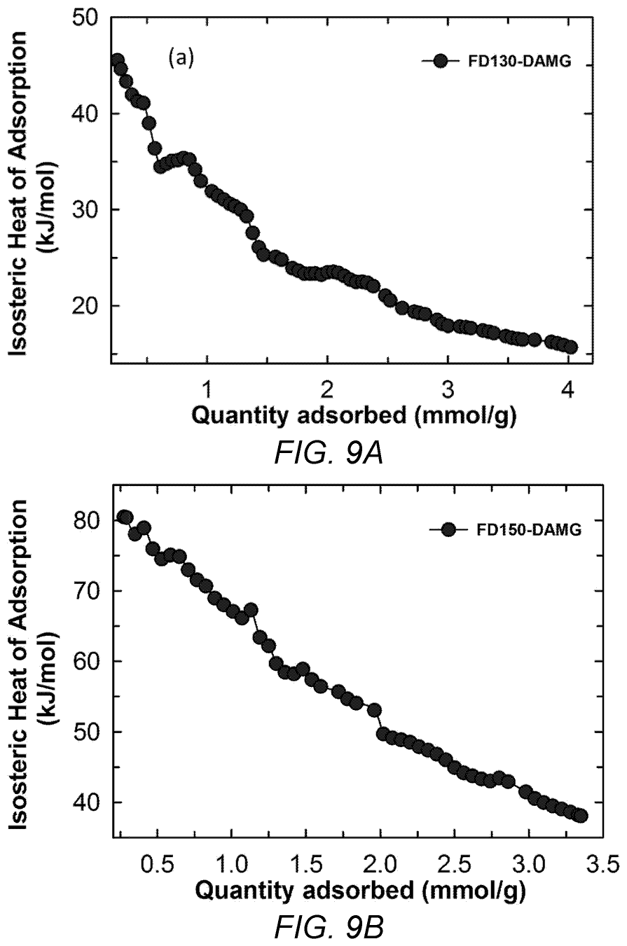

FIG. 11. N₂ adsorption-desorption isotherms of FDU-12-T silica template.

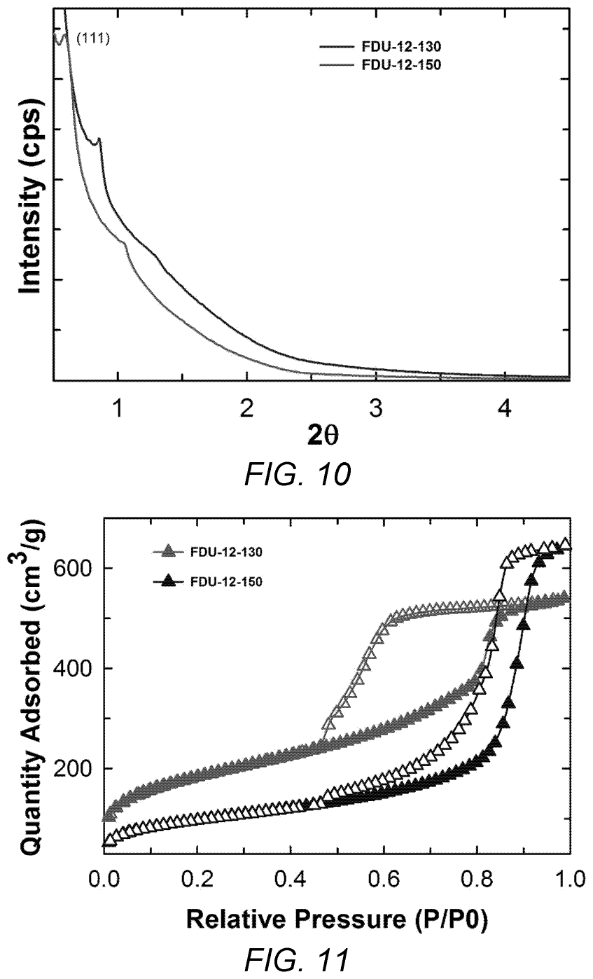

FIGS. 12A-12B. (FIG. 12A) Illustrates a schematic representation of the use of the gMCN-material to capture CO₂. (FIG. 12B) Illustrates a schematic representation of the use of the gMCN material to produce activated CO₂.

### DETAILED DESCRIPTION OF THE INVENTION

Mesoporous carbon nitrides (MCN) were discovered in 2005. Since then a new class of MCN with two- or three-dimensional structure and large pore diameters has been reported. This new class of MCN has potential applications in the fields of catalysis, gas adsorption, and energy conversion due to unique textural features, surface features, optical properties, and electronic properties. In general, MCN materials with different structures and pore diameters can be synthesized using a variety of mesoporous silica as sacrificial templates. More recently, three-dimensional structured MCNs with large pore size, high surface area, and uniform morphology have been reported. However, although the reported MCN materials have showed unique textural parameters for various catalytic performances and gas adsorption capacities, additional MCN with new structures and high nitrogen content is still desired for improving their performance as it relates to CO₂ capture and activation.

Aspects of the invention are directed to compositions including and methods for synthesizing nitrogen rich 3D mesoporous graphitic carbon nitride (3D gMCN) having a stoichoimetric configuration of C₃N_{4.5} with spherical shaped morphology and tunable pore diameters. In certain aspects the gMCN is produced from diaminoguanidine based precursors (e.g., 1,3-diaminioguanidine). In certain aspects the C₃N_{4.5} gMCN possess a 3D Cage type mesoporous structure. The gMCN can possess a BET surface area in the range of 180, 185, 190, 195 to 200 m²/g, in certain aspects 190 to 198 m²/g, and total pore volume of 0.4 to 0.7 cm³/g, in certain aspects 0.5 cm³/g. In a further aspect the gMCN possess an average pore diameter in the range of 6.5-9.5 nm.

The C₃N_{4.5} gMCN can be synthesized using a silica templating approach with the final product being a negative replica of the silica template used. In certain aspects the template is a mesoporous silica FDU-12 having 3D cage type structure. The C₃N_{4.5} gMCN can be synthesized by using diaminoguanidine, such as 1,3-diaminoguanidine. In certain aspects the gMCN has a CO₂ adsorption capacity of 7.0, 7.5, 8.0, 8.5, 9.0, to 9.5 mmol/g, in certain aspects 8.8 mmol/g, at 273 K and 30 bar. In other aspects the gMCN has a very high isosteric heat of adsorption varying the range 12, 15, 20, 25, 30, 35, 40, 45, 50, 55, 60, 65, 70, 75, to 80 kJ/mol, in certain aspects 38-80 kJ/mol, calculated from CO₂ isotherms obtained at 273 K and 283 K using Clauius-Clayperon equation.

Highly ordered 3D cage type mesoporous carbon nitride FD-T-DAMG (DAMG=1,3-diaminoguanidine) with different pore diameters and nitrogen content have been prepared by a hard templating route using 3D cage type FDU-12 silica as the hard template and a new high nitrogen containing diaminioguanidine precursor (e.g., 1,3-diaminoguanidine). The materials were characterized by low and high angle powder XRD, N₂ adsorption-desorption technique, FT-IR, XPS, elemental analysis techniques and XANES. The gMCN materials as illustrated in the non-limiting Examples, can exhibit high structural order and pore diameters tuned from 6.5 nm to 9.5 nm. Elemental analysis shows a very high bulk nitrogen content of nearly 50% and a bulk carbon content of 30%. The elemental analysis shows an N/C ratio in the range of 1.5-1.6, which is much higher than the theoretically predicated ideal C₃N₄ (N/C=1.33). Further FT-IR and XPS studies confirm the presence of residual and terminal —NH and —NH₂ functional groups and high nitrogen content. From XPS survey spectrum, the materials exhibit C₃N_{4.5} stoichiometric configuration. SEM imaging shows a spherical morphology which is confirmation of replication of morphology from the silica FDU-12 template to the carbon nitride.

DFT calculations suggest that defective carbon nitride can chemisorb and activate CO₂ at room and/or mild temperature. In particular, the activation of CO₂ to a bent geometry seems to be feasible in presence of high concentration of primary and secondary amino groups (NH₂ and NH) because of the formation of multiple H-bonds between the molecule and the carbon nitride framework. The computational results suggest also a relatively easy CO₂ desorption process due to moderate binding energy. The identified defect-engineered carbon nitride material seems then to be promising for CO₂ capture as it represents a compromise between the other sorbent materials associated to physical or chemical adsorption mechanism. Based on the computational conclusion, a strategy has been formulated to enhance the number of —NH₂ species and their accessibility.

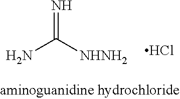

Because polymerization occurs between —NH/—NH₂ species, and —N/—NH species for the aminoguanidine, the number of —NH₂ species should significantly be enhanced by using this monomer.

Typically, mesoporous materials, like SBA-15, KIT-6, and FDU-12 are used as hard templates. The pore volume of those materials is filled by the CN precursors. Then, a thermal treatment is applied to carry out for the polymerization. After this step, the silica template is removed by an appropriate treatment. The morphology of the final material is the replica of the silica mesoporosity. By applying this approach, it is possible to facilitate the accessibility of the —NH₂ species and enhance the CO₂ reactivity.

A. Process for Preparing Nitrogen Rich Three-Dimensional C₃N_{4.5} Mesoporous Graphitic Carbon Nitride (gMCN)

The gMCN material can be formed by using a templating agent. A template can be a mesoporous silica. In one aspect, the mesoporous silica can be an FDU-12 silica material or derivatives thereof.

1. Process to Prepare Template

The silica template can be synthesized under static conditions using a templating approach performed under acidic conditions. The templating agent can be a polymeric compound such as an amphiphilic triblock copolymer of a central hydrophobic block of polypropylene glycol flanked by two hydrophilic blocks of polyethylene glycol. A commercially available amphiphilic triblock copolymer templating agent is available from BASF (Germany) and sold under the trade name Pluronic F127. The silica source can be any suitable silica containing compounds such as sodium silicate, tetramethyl orthosilicate, silica water glass, etc. A non-limiting example of the silica source is tetraethyl orthosilicate (TEOS), which is available from various commercial suppliers (e.g., Sigma-Aldrich®, U.S.A.). An aqueous solution of templating agent (e.g., the amphiphilic triblock copolymer) can be prepared by adding the templating agent to water and stirring the aqueous solution at 20 to 30° C., 23 to 27° C., or 25° C. until the reaction mixture is homogeneous (e.g., 3 to 5 hour). Aqueous mineral acid (e.g., 2 M HCl) can be added to the templating solution to obtain a solution having a pH of 2 or less. After addition of the acid, the temperature of the templating solution can be increased to 35 to 50° C., or 40° C. and agitated for a desired amount of time (e.g., 1 to 5 hours, or 2 hours). The silica source (e.g., TEOS) can be added under agitation to the templating solution for a desired amount of time (e.g., 10 to 30 minutes) and then held (incubated) without agitation (e.g., 24 hours) to form the polymerization solution containing the templating agent and the silica source. The polymerization solution can then be reacted under hydrothermal reaction conditions to form a silica template for a desired amount of time (e.g., 40 to 60 hours, or 45 to 55 hours, or 48 hours). In some embodiments, the reaction conditions can be autogenous conditions. A reaction temperature can range from 100° C. to 200° C., 110° C. to 180° C., 130° C. to 150° C., or any value or range there between. The reaction temperature can be used to tune the pore size of the silica template. By way of example, heating the reaction mixture to 100° C. under autogenous conditions for about 48 h can result in a silica template having a pore size of about 9.12 nm. Increasing the temperature from 100° C. to 130° C. can result in a 10 to 15% increase in pore size (e.g., to 10.5 nm). As the temperature is increased to 150° C., the pore size is further increased by 5 to 10% (e.g., to 11.2 nm, or an overall increase of 15 to 20%, or 18%). Wall thickness of the silica template can also be tuned by the reaction temperature. By way of example, higher reaction temperatures can produce thinner walls.

The silica template can be separated from the polymerization solution using known separation methods (e.g., gravity filtration, vacuum filtration, centrifugation, etc.) and washed with water to remove any residual polymeric solution. In a particular embodiment, the template is filtered hot. The filtered silica template can be dried to remove the water. By way of example, the filtered silica template can be heated at 90 to 110° C. until the silica template is dry (e.g., 6 to 8 hours). The dried filtered silica template can be extracted with alcohol (e.g., ethanol, methanol, propanol, etc.) at 20 to 30° C. (e.g., room temperature and in the absence of external heating or cooling) to remove any residual templating agent (e.g., copolymer and/or polymerized material). In a non-limiting example, the dried filtered silica template can be repeatedly agitated in fresh ethanol solutions until at least 80%, at least 90%, at least 92%, at least 95%, or at least 100% of the templating agent is removed. The ethanol extracted silica template can be dried to remove the alcohol and form a silica template. In certain aspects the silica template can be calcined at a temperature between 500 and 600° C., preferably 540° C. In a particular embodiment, the silica template is mesoporous FDU-12 silica template. The FDU-12 silica template can have a pore diameter ranging from 6 nm to 13 nm, 6.5 nm to 9.5 nm, or 6 nm, 6.1 nm, 6.2 nm, 6.3 nm, 6.4 nm, 6.5 nm, 6.6 nm, 6.7 nm, 6.8 nm, 6.9 nm, 7 nm, 7.1 nm, 7.2 nm, 7.3 nm, 7.4 nm, 7.5 nm, 7.6 nm, 7.7 nm, 7.8 nm, 7.9 nm, 8 nm, 8.1 nm, 8.2 nm, 8.3 nm, 8.4 nm, 8.5 nm, 8.6 nm, 8.7 nm, 8.8 nm, 8.9 nm, 9 nm, 9.1 nm, 9.2 nm, 9.3 nm, 9.4 nm, 9.5 nm, 9.6 nm, 9.7 nm, 9.8 nm, 9.9 nm, 10 nm, 10.1 nm, 10.2 nm, 10.3 nm, 10.4 nm, 10.5 nm, 10.6 nm, 10.7 nm, 10.8 nm, 10.9 nm, 11 nm, 11.1 nm, 11.2 nm, 11.3 nm, 11.4 nm, 11.5 nm, 11.6 nm, 11.7 nm, 11.8 nm, 11.9 nm, 12 nm, 12.1 nm, 12.2 nm, 12.3 nm, 12.4 nm, 12.5 nm, 12.6 nm, 12.7 nm, 12.8 nm, 12.9 nm, or any value there between. A wall thickness of the SBA silica template can range from 0.1 to 3 nm, or 0.3 to 2.8 nm, or 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 nm or any value there between.

2. Process to Prepare an gMCN Material

The gMCN material of the present invention can be prepared using the silica template (e.g., FDU-12) described above and throughout the specification. The silica template pores can be filled with a carbon nitride precursor material(s) to form a template/carbon nitride precursor mixture. By way of example, the FDU-12 silica material can be added to a diaminoguanidine precursor (e.g., 1,3-diaminoguanidine). The template/carbon nitride precursor mixture can be subjected to conditions suitable to form a carbon nitride composite having the shape of the template. The template/carbon nitride mixture can be subjected to an initial incubation at a temperature of 80 to 100° C., or 85 to 95° C., or about 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C. or 100° C., or any value there between. After the initial incubation the mixture is incubated at an increased temperature of 140 to 180° C., preferably 160° C. for 4 to 8 h, or about 6 h. In some embodiments, the solution is refluxed under constant agitation for 5 to 8 hours, or 6 hours, forming a template/carbon nitride (CN) composite. The template/CN composite can be separated from the solution using known separation methods (e.g., distillation, evaporation, filtration, etc.). By way of example, the solution can be removed from the template/CN composite by evaporating the solution under vacuum. The resulting template/CN composite can be dried, and then reduced in size with force (e.g., crushed). Drying temperatures can range from 90 to 110° C., or 100° C.

The dried template/CN composite can be subjected to conditions sufficient to carbonize the material and form a mesoporous carbon nitride material/template complex (e.g., FDU-12 complex). Carbonizing conditions can include a heating the template/CN composite to a temperature of at least 500° C., at least 600° C., at least 700° C., at least 800° C., at least 900° C., at least 1000° C., or 1100° C. In certain aspects, the template/CN composite is heated to 500° C. Notably, the material does not change during carbonization (e.g., the material maintains its shape after it has been carbonized). The nitrogen properties and textural properties of the gMCN material can be tuned by using a specific carbonization temperature. By way of example, the pore diameter of the resulting gMCN material can increase with increasing carbonization temperature up to 900° C. At a temperature of 900° C. or more, the textural properties become saturated and remain substantially unchanged. Nitrogen content can be also be tuned by varying the carbonization temperature. With increasing carbonization temperature, there can be a progressive increase in the C atomic % while there a proportional decrease in the N atomic %. Without wishing to be bound by theory, it is believed that at higher temperatures, N tends to escape from the system by breaking bonds. By way of example, a template/CN composite heated at 600° C. can have an N atomic % of about 16%, and after heating at 1100° C. have a N atomic % of about 3%. The carbon content can also be tuned based on a selected temperature as the atomic carbon content increases as the temperature rises. By selecting a desired carbonization temperature, the C/N atomic ratio of the mesoporous carbon nitride material of the present invention can be tuned. In one particular embodiment, a carbonization temperature of 500° C. provides a nitrogen to carbon (N/C) ratio of about 1.45 to 1.6. In some embodiments the ratio is 1.5.

The template can be removed from the carbonized material (e.g., the mesoporous carbon nitride material/template composite) by subjecting it to conditions sufficient to dissolve the template and form the mesoporous carbon nitride material of the present invention. By way of example, the template can be dissolved using an hydrofluoric acid (F) treatment, a very high alkaline solution, or any other dissolution agent capable of removing the template and not dissolving the CN framework. The kind of template and the CN precursor used influence the characteristics of the final material. The resulting gMCN material of the present invention can be washed with solvent (e.g., ethanol) to remove the dissolution material, and then dried (e.g., heated at 100° C.).

B. Graphitic Mesoporous Carbon Nitride Materials (gMCN)

The gMCN material can have an average pore size or pore diameter of 6 nm, 6.5 nm, 7 nm, 7.5 nm, 8.0 nm, 8.5 nm, 9.0 nm, or 9.5 nm. Specifically the pore size can range from 6.5 to 9.5 nm, or about 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, or 9.5 nm. The pore volume of the mesoporous material can range from 0.4-0.7 cm³/g or any value or range there between (e.g., 0.40, 0.41, 0.42, 0.43, 0.5, 0.51, 0.52, 0.53, 0.54, 0.55, 0.56, 0.57, 0.58, 0.59, 0.60, 0.61, 0.62, 0.63, 0.64, 0.65, 0.67, 0.68, 0.69, or 0.70 cm³g⁻¹). Preferably, the pore volume is 0.5 cm³g⁻¹. The BET surface area of the can be from 180 to 200 m²/g, preferably 190 to 198 m²/g. In certain embodiments a gMCN material is made from a silica template prepared at 130° C. or 150° C., or any temperatures or range of temperatures there between.

### C. Use of the Mesoporous Carbon Nitride Materials

The gMCN materials of the present invention can be used in applications for sequestration or activation of carbon dioxide. Certain embodiments of the invention are directed to systems for CO₂ sequestration, capture, and activation.

According to one embodiment of the present invention, a process for CO₂ capture is described. In step one of the process, a feed stock comprising CO₂ is contacted with gMCN. The feed stock can include a concentration of CO₂ from 0.01 to 100% and all ranges and values there between (e.g., 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.22, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.50, 0.51, 0.52, 0.53, 0.54, 0.55, 0.56, 0.57, 0.58, 0.59, 0.60, 0.61, 0.62, 0.63, 0.64, 0.65, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%). The % of CO₂ in the feed stock can be measured in wt. % or mol. % or volume % based on the total wt. or mol. or volume of the feed stock respectively. In a preferred aspect, the feedstock can be ambient atmospheric or a gas effluent from a CO₂ producing process. In one non-limiting instance, the CO₂ can be obtained from a waste or recycle gas stream (e.g., a flue gas emission from a power plant on the same site such as from ammonia synthesis or a reverse water gas shift reaction) or after recovering the carbon dioxide from a gas stream. A benefit of recycling carbon dioxide as a starting material can reduce the amount of carbon dioxide emitted to the atmosphere (e.g., from a chemical production site). The feedstock containing CO₂ can contain additional gas and/or vapors (e.g., nitrogen (N₂), oxygen (O₂), argon (Ar), chloride (C₁₂), radon (Ra), xenon (Xe), methane (CH₄), ammonia (NH₃), carbon monoxide (CO), sulfur containing compounds (RxS), volatile halocarbons (all permutations of HFCs, CFCs, and BFCs), ozone (O₃), partial oxidation products, etc.). In some examples, the remainder of the feedstock gas can include another gas or gases provided the gas or gases are inert to CO₂ capture and/or activation for further reaction so they do not negatively affect the gMCN material. In instances where another gas or vapor do have negative effects on the CO₂ capture process (e.g., conversion, yield, efficiency, etc.), those gases or vapors can be selectively removed by known processes. Preferably, the reactant mixture is highly pure and substantially devoid of water. In some embodiments, the CO₂ can be dried prior to use (e.g., pass through a drying media) or contain a minimal amount of water or no water at all. Water can be removed from the reactant gases with any suitable method known in the art (e.g., condensation, liquid/gas separation, etc.).

The process can further comprise, holding the reactant mixture (incubating) under conditions in which CO₂ is attached to the mesoporous material. For example, the CO₂ can be adsorbed to the mesoporous material or can covalently bind to a primary or secondary nitrogen group of the mesoporous material. The incubation conditions can include a temperature, pressure, and time. The temperature range for the incubation can be from 0° C. to 30° C., from 5° C. to 25° C., 10° C. to 20° C., and all ranges and temperatures there between. The pressure range for the incubation can be from 0.1 MPa to 3 MPa, or 1 to 2 MPa. In embodiments, where adsorption/desorption processes are used, the pressure of adsorption is higher than a pressure of desorption. By way of example, a gas including methane, hydrogen, or other less adsorbing gases, the adsorbing CO₂ partial pressure can range from 0.1 to 3 MPa and the desorbing CO₂ partial pressure can range from 0 MPa to 2 MPa. The time of incubation can be from 1 sec to 60 seconds, 5 minutes to 50 minutes, 10 minutes to 30 minutes. The conditions for CO₂ capture can be varied based on the source and composition of feed stream and/or the type of the reactor used.

According to another embodiment of the current invention, the gMCN material containing attached CO₂, the CO₂ can be released to regenerate the gMCN material and release CO₂. Without limitation, equilibrium binding between the gMCN material and CO₂ can occur. In some aspects, an equilibrium binding constant can be determined and influenced by typical reaction condition manipulations (e.g., increasing the concentration or pressure of the reactant feed stock, etc.). The methods and system disclosed herein also include the ability to regenerate used/deactivated gMCN in a continuous process. Non-limiting examples of regeneration include a pressure swing adsorption (PSA) process at a lower pressure and/or a using a change of feed material. In some embodiments, the gMCN/CO₂ is disposed in an environmentally safe manner.

Certain embodiments of the invention are directed to systems for CO₂ capture. In general aspects, a first stage of a system for CO₂ capture includes moving a flowing mass of ambient air having the usual relatively low concentration of CO₂ in the atmosphere, with a relatively low pressure drop (in the range of 100-1000) pascals. The flow of C₂ containing air from the first stage, can be passed, in a second stage, through a large area bed, or beds, of sorbent (e.g., including a gMCN of the present invention) for the CO₂, the bed having a high porosity and on the walls defining the pores a highly active CO₂ adsorbent.

In general aspects, the first stage of a system for CO₂ capture includes moving a flowing mass of ambient air having the usual relatively low concentration of CO₂ in the atmosphere, with a relatively low pressure drop (in the range of 100-1000) pascals. The flow of CO₂ containing air from can be passed through a large area bed, or beds, of sorbent (e.g., including gMCN) for the CO₂, the bed having a high porosity and on the walls defining the pores a highly active CO₂ adsorbent.

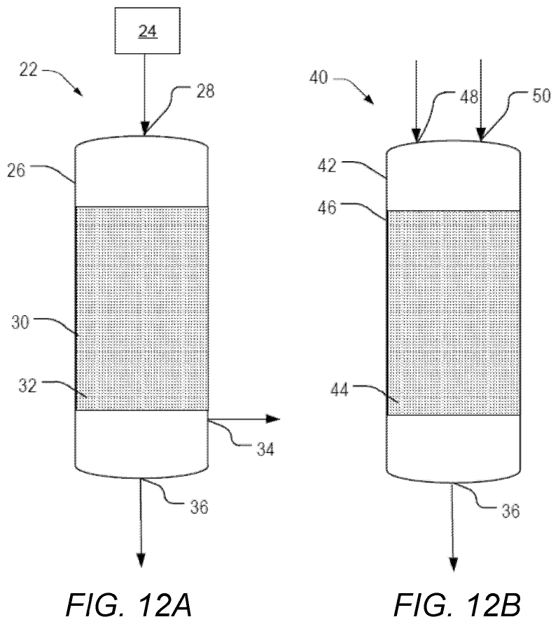

Other embodiments include systems for CO₂ capture and activation to form a reaction product. Referring to FIG. 12A and FIG. 12B, systems are illustrated, which can be used to capture CO₂ using the gMCN material of the present invention and/or activate the CO₂. The system 22 can include a feed source 24, a separation unit 26. The feed source 24 can be configured to be in fluid communication with the separation unit 26 via an inlet 28 on the separation unit. The feed source can be configured such that it regulates the amount of CO₂ containing material entering the separation unit 26. The separation unit 26 can include at least one separation zone 30 having the gMCN material 32 of the present invention. Although not shown, the separation unit may have additional inlets for the introduction of gases that can be added to the separation unit as mixtures or added separately and mixed within the separation unit. Optionally, these additional inlets may also be used as an evacuation outlet to remove and replace the atmosphere within the separation unit with inert atmosphere or reactant gases in pump/purge cycles. To avoid the need to remove atmosphere from the separation unit, the entire separation unit can kept under inert atmosphere. The separation unit 26 can include an outlet 34 for uncaptured gases in the separation unit. The separation unit can be depressurized or chemically treated to remove the desorbed or bound CO₂ from the gMCN material. A second unit can be used in combination with separation unit 26 to provide a continuous process. The released CO₂ can exit the separation unit from outlet 36 and be collected, stored, transported, or provided to other processing units for further use.

Referring to FIG. 12B, system 40 is system used to activate CO₂ for use in producing alcohols or carbonylated materials. Reactor 42 can include gMCN material 44 in reaction zone 46. CO₂ can enter reactor 42 via inlet 48 and an olefinic (e.g., olefin, substituted olefin, aromatic, substituted aromatic compound) can enter reactor 42 via inlet 50. The CO₂ and olefinic material can mix in reactor 42 to form a reactant mixture. In some embodiments, the CO₂ and olefinic material can be provided as one stream to reactor 42. In reaction zone 46 as the CO₂ and olefinic material pass over the gMCN material, the basic nitrogen sites on the gMCN material can activate or bond to the CO₂ and promote addition of an oxygen and/or a CO to the olefinic compound. By way of example, CO₂ and benzene can be contacted with the gMCN material to produce phenol and CO. The reactor 42 can be heated under desired pressures and temperatures to promote the reaction of CO₂ with the olefinic material. The reaction product can exit reactor 42 via product outlet 52 and be collected, stored, transported, or provided to other units for further processing. If necessary, the reaction product can be purified. For example, unreacted CO₂ and olefinic compound can be separated (e.g., separation system 22) and recycled to reactor 42. Systems 22 and 40 can also include a heating source (not shown). The heating source can be heaters, heat exchange systems or the like, and be configured to heat the reaction zone 42 or separation zone 4 to a temperature sufficient to perform the desired reaction or separation.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation and Characterization of Graphitic Mesoporous Carbon Nitrides

Preparation of FDU-12-T (T=130, 150° C.) silica templates. In a typical synthesis of FDU-12, 2 g of F127 is mixed with 3 g KCl and 2 g 1,3,5-trimethylbenzene to which 120 g of 2M HCl was added and stirred for 4 h at room temperature. After this, 8.3 g of tetraethyl orthosilicate (TEOS) was added slowly with constant stirring and the temperature was increased to 35° C. The resulting mixture for stirred at 35° C. for 24 h and then transferred to a Teflon lined stainless streel autoclave which is then transferred to an oven at 130/150° C. for a period of 24 h. The product was filtered in hot and washed with water once to remove KCl salt, followed by drying in air at 100° C. for 6 h. The polymeric surfactant F127 was removed by calcination at 540° C. under air/nitrogen environment. Following the above procedure, FDU-12-T (T=hydrothermal synthesis temperature) samples were prepared at 130 and 150° C. and labelled as FDU-12-130 and FDU-12-150.

Preparation of 3D Cage type MCN using 1,3-diaminoguanidine (DAMG) as the precursor. FD-T-DAMG (T=temperature, DAMG=1,3 diaminoguanidine) was prepared using a hard templating approach using FDU-12 as the silica template and 1,3 diaminoguanidine as the single molecular carbon and nitrogen precursor. In a typical synthesis, 4 g of 1,3 diaminoguanidine (DAMG) was dissolved in 5 g of DI water. The resulting solution was heated at 60° C. in a water bath or an oven for few minutes till a clear solution is obtained. The resulting solution was quickly poured onto 1 g of silica template FDU-12-130/150 and mixed thoroughly for about 15 minutes by applying sufficient pressure (hand pressure only). After ensuring thorough mixing, the resulting pasty mixture was kept in an oven at 100° C. for 6 h and then the temperature was increased to 160° C. and maintained for another 6 h. The resulting white color composite was crushed in a mortar and pestle and kept at the center of an alumina boat and carbonized in a tubular furnace at 400° C. for 5 h with a heating rate of 3° C./min under nitrogen/argon environment. The carbonized sample was then treated with 5 wt. % aqueous solution of hydrofluoric acid to dissolve the silica template and recover porous carbon nitride. The dark yellow powered sample was washed with excess ethanol and dried at 100° C. for 6 h before characterization.

Materials Characterization. The silica templates FDU-12-T (T is the hydrothermal temperature T=130 and 150° C.) and the corresponding carbon nitrides FD-T-DAMG (DAMG=1,3-diaminoguanidine) were characterized with low angle powder XRD. The Powder X-ray diffraction measurements were carried out on a PANalytical Empyream platform diffractometer using Bragg-Brentano geometry. The measurements were collected using Cu K_{α} radiation from a sealed tube source operating at 40 kV and 40 mA, a fixed divergence slit of 0.1 degree and a PIXcel^{3D} detector. The scan rate used was 0.01 degree/sec. The low angle measurements were done in the 2 Theta range 0.1 degree to 5 degree and wide angle measurements were from 5 degree to 70 degree. Nitrogen adsorption and desorption isotherms were measured at −196° C. on a Micromeritics ASAP 2420 surface area and porosity analyzer. All the samples were degassed for 8 h at 250° C. under a vacuum (p<1×10⁻⁵ pa) in the degas port of the adsorption analyzer. The specific surface area was calculated using the standard BET model. Pore size distribution was obtained from the adsorption branches of the nitrogen isotherms using the BJH model. FT-IR spectra were recorded on Nicolet Magna-IR 750 fitted with a MTEC Model 300 Photoacoustic measuring 256 scans, at a resolution of 8 cm⁻¹, and a mirror velocity of 0.158 cm/s which equates to a sampling depth of ˜22 microns.

XPS data was acquired using a Kratos Axis ULTRA X-ray Photoelectron Spectrometer incorporating a 165 mm hemispherical electron energy analyzer. The incident radiation was Monochromatic Al Kα X-rays (1486.6 eV) at 225 W (15 kV, 15 ma). Survey (wide) scans were taken at analyzer pass energy of 160 eV and multiplex (narrow) high resolution scans at 20 eV. Survey scans were carried out over 1200-0 eV binding energy range with 1.0 eV steps and a dwell time of 100 ms. Narrow high-resolution scans were run with 0.05 eV steps and 250 ms dwell time. Base pressure in the analysis chamber was 1.0×10⁻⁹ Torr and during sample analysis 1.0×10⁻⁸ Torr. Atomic concentrations were calculated using the CasaXPS version 2.3.14 software and a Shirley baseline with Kratos library Relative Sensitivity Factors (RSFs). Peak fitting of the high-resolution data was also carried out using the CasaXPS software. The structural morphology of the samples was observed in JEOL FE SEM 7001. The sample preparation for HR-SEM involved sprinkling of a small quantity of powder sample on the carbon tab. The stub is kept in a vacuum oven at 70° C. for 7 h before insertion into the SEM. The samples were coated with 5 nm layer of Iridium using Baltek coater using a nominal current of 15.5 mAps and coating time 60 sec. High pressure CO₂ adsorption was carried out on Quanta chrome Isorb HP1 equipped with temperature controlled circulator. The CO₂ adsorption was carried out at 30 bar and different analysis temperatures 273 K was used. Prior to CO₂ adsorption, samples were degassed for 10 h at 250° C. The strength of interaction between MCN and CO₂ molecules was calculated using Clausius-Clayperon equation.

### Results and Discussion

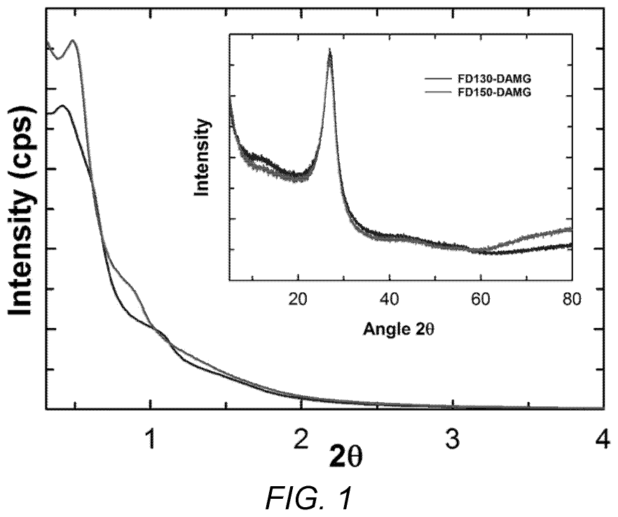

X-ray Diffraction. FIG. 1 shows the low angle XRD patterns of FD-T-DAMG samples and inset shows the wide angle patterns for these samples. Both the samples show two distinct peaks in the low angle XRD. One is lower order sharp peak and another higher order peak. From the low angle XRD plots, it is clear that both the carbon nitride sample exhibit well-defined structural ordering. Between the two samples, the peak intensity of FD-150-DAMG is slightly higher than that of FD-130-DAMG. Inset shows the wide angle plots for these samples. Interestingly, wide angle plot for both the samples exactly overlap suggesting almost similar extent of graphitization in both the samples. Further, the plots show only one sharp peak at 2-theta=27 degrees, which is indicative of interplanar stacking of CN layers. However, the lower angle reflection is missing. Further, since both the samples have almost identical peak intensity at 2-theta=27 degrees, the results suggest that both the samples have almost equal degree of graphitization.

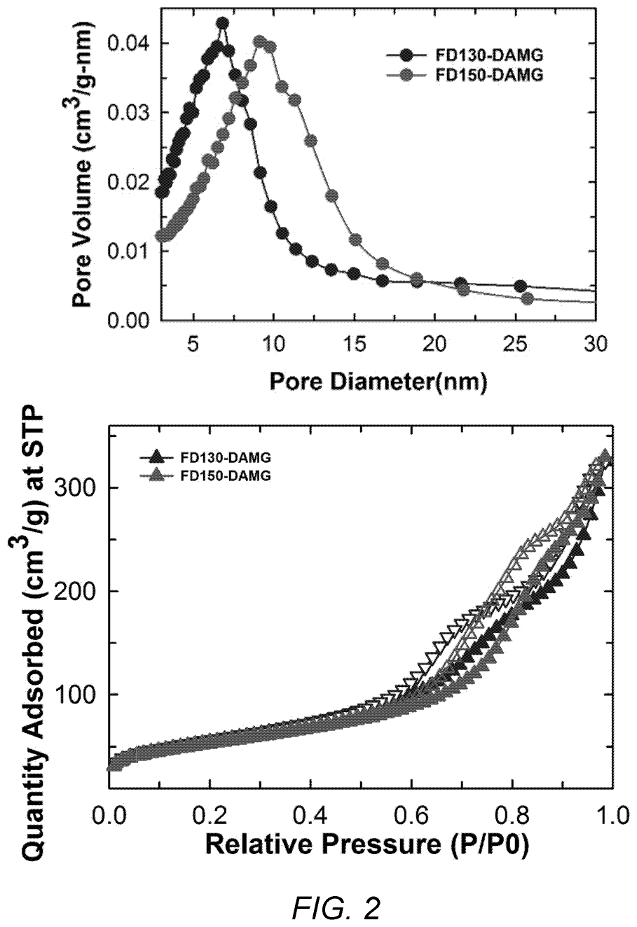

N₂ Adsorption-desorption. FIG. 2 shows the nitrogen adsorption-desorption isotherms for FD-T-DAMG samples and the inset shows the pore size distribution for these samples. From FIG. 2, it is clear that these materials exhibit type IV isotherm which as per the IUPAC convention is typically associated with mesoporous materials. It is interesting to note that the BET surface area and pore volumes of both the sample are almost identical as shown in Table 1, however, the pore diameters are different. Changing the pore diameter also influences other textural parameters namely BET surface area and pore volume, however in this case, it appears that there was no effect on the textural parameters with change in the pore diameter. The pore diameter of FD-130-DAMG is less than that of FD-150-DAMG, which is an expected result since FD-130-DAMG is prepared using a similar pore diameter silica template FDU-12-130 whereas FD-150-DAMG is prepared using a larger pore diameter template FDU-12-150. The pore size distribution of these samples is shown in the inset and shows a broad peak centered at 6.8 nm and 9.3 nm for FD-130-DAMG and FD-150-DAMG respectively.

TABLE 1
Textural parameters, CO₂ adsorption and elemental
composition of FD-T-DAMG samples.

| | | | | ^{#}CO₂ | | XPS | | | CHN | | |
|---|---|---|---|---|---|---|---|---|---|---|---|
| | S.A | P.D | P.V | (mmol/g) | | (%) | | | (%) | | |
| Sample | (m²/g) | (nm) | (cm³/g) | 273K | 283K | C | N | O | C | N | H |
| FD-130-DAMG | 198 | 6.8 | 0.5 | 7.2 | 4 | 39.5 | 58 | 2.1 | 31.2 | 48 | — |
| FD-150-DAMG | 190 | 9.3 | 0.5 | 8.8 | 3.3 | 39 | 59 | 1.6 | 32 | 51 | — |

^{#}CO₂ adsorption was done using dry CO₂ gas up to 30 bar.

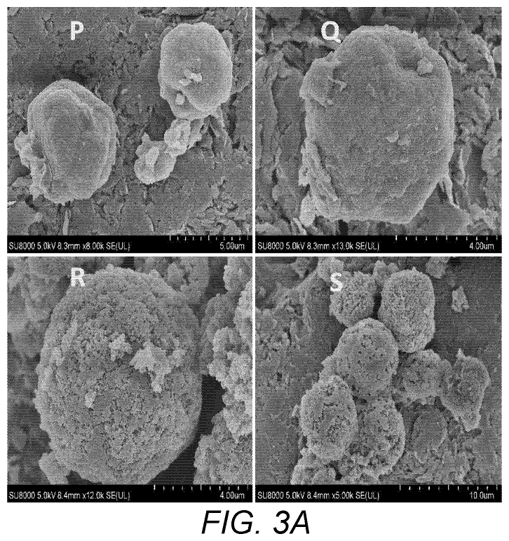

Electron Microscopy imaging—SEM and TEM. FIG. 3A shows the SEM images of FD-T-DAMG samples which show a very uniform spherical morphology. The results show a successful replication of spherical morphology of the template FDU-12 to the corresponding nitrides. FDU-12 is known to have spherical particles as reported in literature. FIG. 3B shows the HR-TEM images of (A1, A2) FD-130-DAMG and (B1, B2) FD-150-DAMG samples clearly showing the presence of mesochannels confirming the results from N₂ sorption and low angle XRD experiments. The TEM images were taken both in the direction of the pore channels as well perpendicular to the pores.

Elemental Analysis. The carbon, nitrogen content of the samples was analysed using the CHN analyser. As shown in Table 1 below, both the samples exhibit nearly 50% Nitrogen content and about 30% carbon content. Interestingly, the bulk composition of the two materials is almost identical. It is to be noted here that although the pore diameters of the silica templates are different but the quantity of precursor impregnated is the same and identical conditions are used for carbonization and silica framework removal, so in theory, the composition of the samples should be nearly same. However, the difference in the pore diameters of the silica template for the same quantity of precursor should result in different wall thicknesses which is clearly manifested in the slight variation in the colors of these two samples.

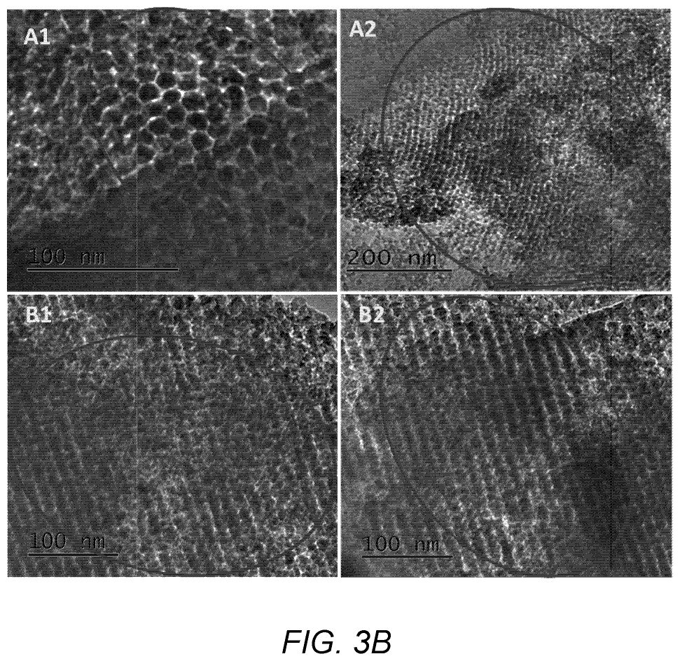

FT-IR. The FT-IR spectrum of FD-130-DAMG is shown in FIG. 4. The plots shows a number vibration bands which were assigned to different functional groups. The peak at 743 cm⁻¹ is characteristic of syn-phase and anti-phase vibration of N═N of the tetrazine ring. Whereas the band at 1328 cm⁻¹ and 1435 cm⁻¹ are attributed to C═N and N═N stretching bonds. The band at 1575 is ascribed to the aromatic ring modes while bands at 3188 and 3354 cm⁻¹ were attributed to —NH and —NH₂ groups. These results are in strong agreement with the XPS analysis.

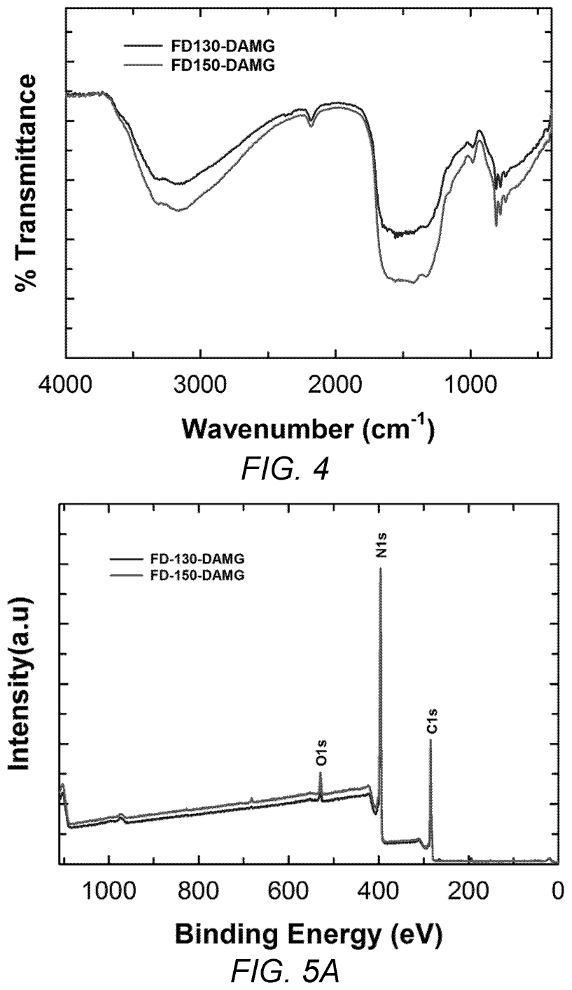

X-ray Photoelectrospectroscopy. The surface atomic distribution of C, N, and O oxygen atoms was investigated by recording the survey spectra of these samples as shown in Table 1 above and in FIG. 5A. From the survey spectra, it is obvious that the surface of these samples have more N atoms than carbon atoms with a very small quantity of surface oxygen atoms. Also for both the samples, the surface spectra nearly overlap suggesting that pore diameter tuning does not alter the surface atomic composition of the materials. The surface composition follows the similar pattern as seen in the bulk elemental analysis. However, it is to be noted that XPS being a surface technique measures atomic composition up to a depth of 10 nm from the top exposed surface. Consequently, it is possible to have higher concentration of C and N atoms because of segregation of atoms within the top 10 nm layer of material and so the results from survey spectra may not be in complete agreement with the bulk elemental compositions.

The nature and co-ordination of C and N was investigated using high resolution N1s and C1s spectra as shown in FIG. 5B and FIG. 5C respectively and the deconvoluted peaks are shown in Table 2 below. The N1s spectra in FIG. 5B was deconvoluted into four peaks. The peak at 398.3 eV was assigned to C—N═C, the peak at 400.1 eV was assigned to nitrogen trigonally bonded to three other carbon atoms (N—C₃), the peak at 401.4 eV was assigned to C—N—H groups and the peak 403.5 eV was assigned to π-π* bond. The C1s spectra in FIG. 5C was deconvoluted into 4 peaks and assigned to different bonding groups as shown as shown in Table 2. The peak at 287.7 eV was assigned to C—N═C, the peak at 284.6 eV was assigned to C═C and the peak at 289.1 eV was assigned to C—N—H bonding groups while the peak at 293.1 eV was assigned to π-π*. The relative percentages of different functional groups identified via XPS is shown in Table 2.

TABLE 2
XPS deconvoluted peaks of C1s and N1s high
resolution spectra of FD-T-DAMG samples

| | C—N═C | C═C | C—N—H | π- π* |
|---|---|---|---|---|
| Sample | 287.7 eV | 284.6 eV | 289.1 eV | 293.1 eV |
| FD-150- | 77.8% | 14.5% | 6.6% | 1.1% |
| DAMG | | | | |
| FD-130- | 67.5% | 23.4% | 7.7% | 1.4% |
| DAMG | | | | |
| | C—N═C | N—(C)₃ | C—N—H | π- π* |
| Sample | 398.3 eV | 400.1 eV | 401.4 eV | 403.5 eV |
| FD-150- | 76.6% | 16.0% | 6.7% | 0.7% |
| DAMG | | | | |
| FD-130- | 65.0% | 26.3% | 7.6% | 1.1% |
| DAMG | | | | |

TABLE 3
Textural parameters of FDU-12-T silica template

| | S.A | P.D | P.V |
|---|---|---|---|
| Sample | (m²/g) | (nm) | (cm³/g) |
| FDU-12-130 | 660 | 11.3 | 0.83 |
| FDU-12-150 | 354 | 16.3 | 0.99 |

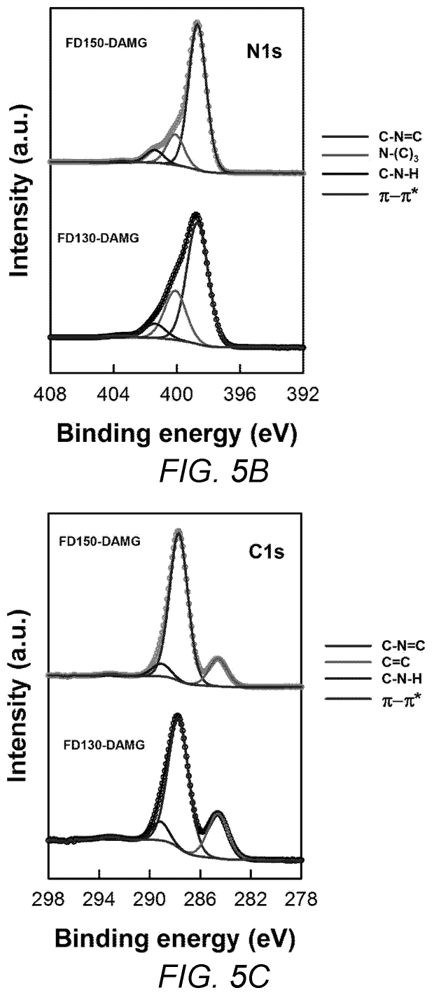

Near Edge X-ray Absorption Fine Structure (NEXAFS). Synchroton based NAXAFS spectra was recorded for the FD150-DAMG sample to gain further insights into the chemical bonding of C and N in the sample as shown in C K-edge (FIG. 6A) and N K-edge (FIG. 6B) for FD150-DAMG sample. From FIG. 6A, it can be seen that the characteristic resonances of graphitic carbon nitride occur at different photo energy values such as π*_{C═C} (C1) at 285.6 eV, π*_{C—N—C} (C2) at 288.0 eV, σ*_{C—C} (C3) at around 294 eV, and structural defects. From FIG. 6B, two typical π* resonances can be observed occurring at photon energies of 399.4 and 402.3 eV, which correspond to aromatic C—N—C coordination in one tri-s-triazine heteroring (N1) and N-3C bridging among three tri-s-triazine moieties (N2), respectively. In comparison to the non-porous graphitic C₃N₄ sample, FD150-DAMG show well pronounced graphitic bonding characteristics.

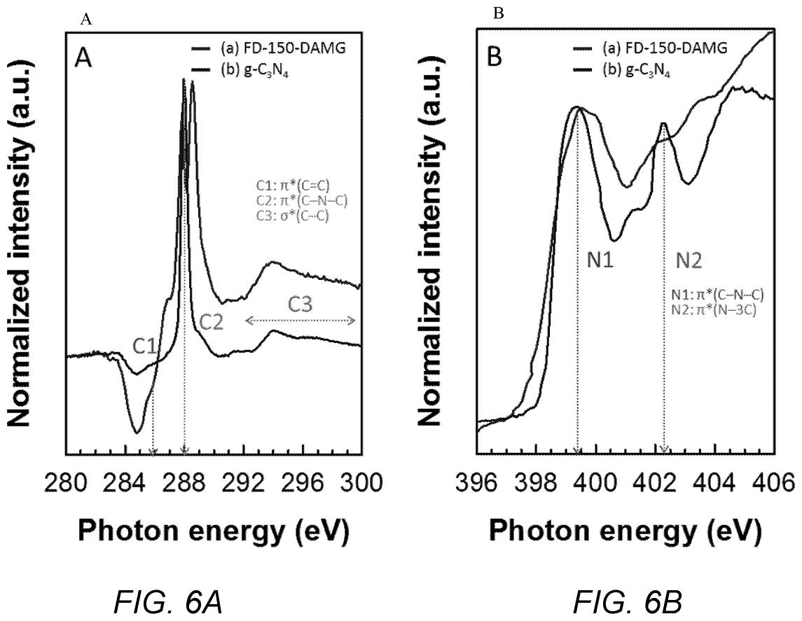

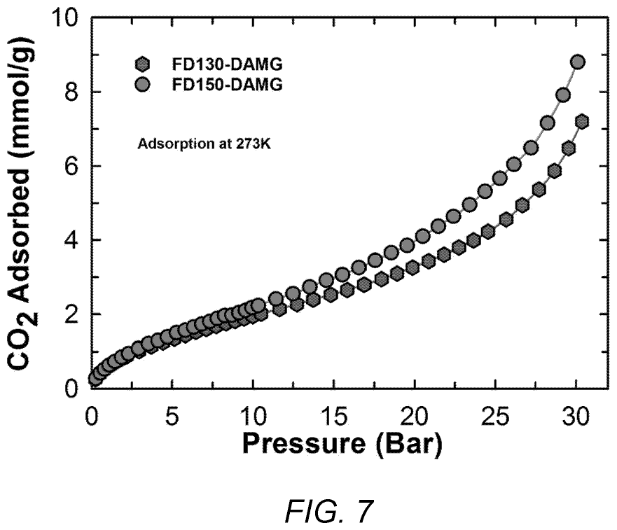

CO₂ adsorption. The MCN samples FD-T-DAMG were used as adsorbed for CO₂ at two different temperatures of 273 K and 283 K and pressure up to 30 bar. The CO₂ adsorption isotherms for the FD-T-DAMG samples are shown in FIG. 7. The CO₂ adsorption capacity of these materials are 7.2 and 8.8 mmol/g for FD-130-DAMG and FD-150-DAMG samples respectively at 273 K and 30 bar. For materials with not very high surface area and high nitrogen content, the adsorption capacity is remarkably impressive in comparison with mesoporous carbon prepared with controlled morphology and has a high surface area of 1200 m²/g shows a CO₂ adsorption capacity of 24.5 mmol/g at 273 K and 30 bar pressure. Interestingly both the materials show nearly the same CO₂ adsorption capacity. CO₂ adsorption on a porous material is mainly dependent on the BET surface area and the presence of basic sites or basic functional moieties. However, it has been found that CO₂ adsorption is dictated by a combination of these two factors. One single factor does not dictate CO₂ adsorption capacity. In the present case, the BET surface areas of the two materials is in the similar range and the bulk nitrogen composition is also nearly same. Based on these, it stands to reason that the CO₂ adsorption capacity would also be nearly same. The effect of analysis temperature on the CO₂ adsorption capacity of these materials was evaluated by recording the CO₂ adsorption isotherms at 283 K and 30 bar as shown in FIG. 8A and FIG. 8B. From the plots, it is clear that the adsorption capacity decreases drastically when the analysis temperature is changed from 273 K to 283 K, the adsorption capacity is less than half when the analysis temperature is increased by 10° C. From this result, it can inferred that the CO₂ adsorption process on FD-T-DAMG samples is an exothermic process and adsorption capacity decreases with increasing analysis temperature.

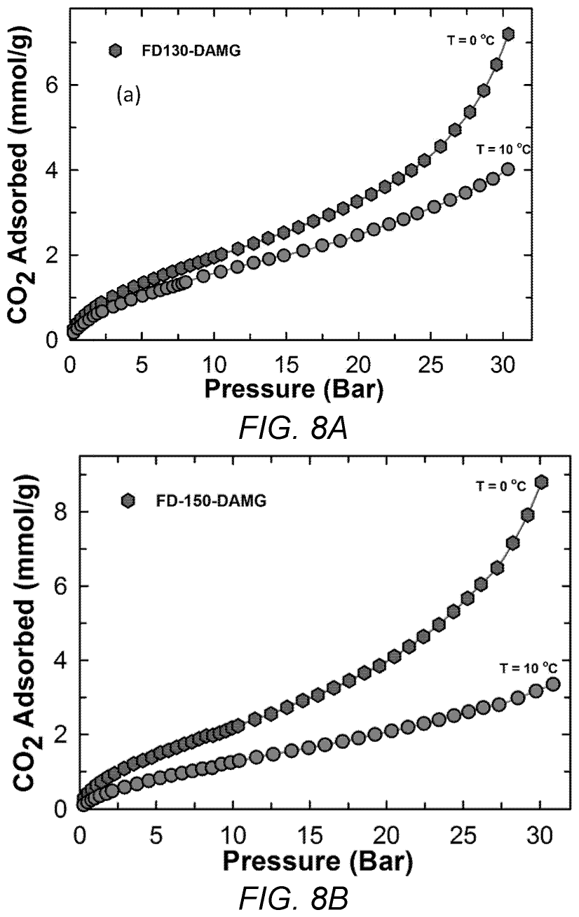

Isosteric heat of adsorption. The strength of interaction between the adsorbate and adsorbent was quantified by calculating the isosteric heat of adsorption of these samples using the isotherms recorded at different analysis temperatures via Clausius-Clayperon equation. The isosteric heat of adsorption for FD-DAMG samples is shown in FIG. 9A and FIG. 9B. For FD-150-DAMG sample, the isosteric heat of adsorption shows a progressive decrease with increasing CO₂ loading and varies in the range 38-80 kJ/mol suggesting a very strong interaction between FD-150-DAMG sample and CO₂ molecules which is also confirmed from that fact that FD-150-DAMG sample shows higher CO₂ adsorption capacity than FD-130-DAMG at 273 K and 30 bar pressure. The isosteric heat of adsorption for FD-130-DAMG sample was found to vary in the range 14.9-45.4 kJ/mol which is much smaller compared to that for FD-150-DAMG sample.

The inventors have successfully demonstrated the synthesis of 3D cage type high nitrogen containing mesoporous carbon nitride with different pore diameter from FDU-12 cage type silica as the hard template and nitrogen rich 1,3-diaminoguanidine as the carbon and nitrogen precursor. The materials showed excellent CO₂ adsorption capacity of 7.2 and 8.8 mmol/g for FD-130-DAMG and FD-150-DAMG respectively which is a highly impressive result since the surface area of these materials is in the range 190-198 m²/g but a very high nitrogen content. Further, the isosteric heat of adsorption was found to vary in the range 38-80 kJ/mol for the FD-150-DAMG sample suggesting very strong interaction between the FD-150-DAMG sample and CO₂ and their suitability for CO₂ capture.

1. A nitrogen rich three-dimensional graphitic mesoporous carbon nitride (gMCN) material having (i) a spherical morphology, (ii) a C₃N₄₊ stoichiometry where the nitrogen to carbon (N/C) ratio from 1.45 to 1.6, and (iii) a monomodal pore distribution with an average pore diameter between 6.5 to 9.5 nm.

2. The material of claim 1, wherein the N/C ratio is 1.5.

3. The material of claim 1, wherein the gMCN is formed from templated diaminoguanidine.

4. The material of claim 1, wherein the material has a BET surface area of 180 to 200 m²/g.

5. The material of claim 1, wherein the material has a total pore volume of 0.4-0.7 cm³/g.

6. The material of claim 1, wherein the material has a CO₂ adsorption capacity of 7.0 to 9.5 mmol/g at 273K and 30 bar.

7. The material of claim 1, wherein the material has an isosteric heat of adsorption of 10, 15, 20, 25, 30, 35 to 40, 45, 50, 55, 60, 65, 70, 75, 80 kJ/mol.

8. The material of claim 1, wherein the material is a negative replica of a FDU-12 silica template.

9. A method of synthesizing a three dimensional carbon nitride material formed from a diaminoguanidine precursor comprising:
(a) contacting a silica template with an aqueous diaminoguanidine precursor solution forming a templated reaction mixture;
(b) heating the templated reaction mixture to a temperature between 40 and 200° C., preferably between 80 and 120° C. for 4 to 8 hours forming a first heated reaction mixture;
(c) heating the first heated reaction mixture to a temperature between 100 and 200° C., preferably between 140 to 180° C., preferable 160° C., for 4 to 8 hours forming a second heated reaction mixture;
(d) carbonizing the second heated reaction mixture by heating to about 300 to 500° C., preferably 400° C., for 4 to 6 hours forming a template/1,3-diaminoguanidine-based carbon nitride product; and
(e) removing the template to form the nitrogen rich three-dimensional C₃N₄₊ graphitic mesoporous carbon nitride (gMCN) material of claim 1.

10. The method of claim 9, wherein the silica template is formed by:
(f) adding tetraethyl orthosilicate (TEOS) to a mixture of F-127 surfactant, potassium chloride (KCl), 1,3,5-trimethylbenzene, and hydrogen chloride (HCl) forming a template reaction mixture;
(g) incubating the template reaction mixture at a temperature of about 30 to 40° C., preferably 35° C. for 1 to 4 hours;
(h) heating the template reaction mixture to 100-200° C. for 1 to 4 days forming a heated template reaction mixture;
(i) drying the heated template reaction mixture at 100° C. for 5 to 10 hours forming a dried template reaction mixture; and
(j) calcining the dried template reaction mixture at a temperature of 500 to 600° C., preferably 540° C., forming a FDU-12 silica template.

11. The method of claim 9, wherein the template reaction mixture is heated at a temperature of about 130° C. forming a FDU-12-130 template.

12. The method of claim 9, wherein the template reaction mixture is heated at a temperature of about 150° C. forming a FDU-1-150 template.

13. The method of claim 9, further comprising crushing the second heated reaction mixture prior to the carbonizing.

14. The method of claim 9, further comprising bringing the second heated mixture to carbonization temperature using a ramping rate of 2 to 4° C./min.

15. The method of claim 9, wherein carbonizing is performed under constant nitrogen flow.

16. The method of claim 9, wherein the first heated reaction mixture is incubated at a temperature of 130° C.

17. The method of claim 9, wherein the first heated reaction mixture is incubated at a temperature of 150° C.

18. The method of claim 9, wherein the template is removed by treating the template/diaminoguanidine-based carbon nitride product with hydrogen fluoride or an ethanol wash.

19. A CO₂ capture process comprising contacting a nitrogen rich three-dimensional C₃N₄₊ graphitic mesoporous carbon nitride (gMCN) of claim 1, with a CO₂ containing feed source, wherein CO₂ is absorbed in or to gMCN.

20. The process of claim 19, further comprising incubating the CO₂ absorbed gMCN under conversion conditions forming a CO₂ conversion product.

21. The process of claim 20, wherein the CO₂ conversion product comprises methanol.
